(19) United States
(12) Patent Application Publication
(10) Pub. No.: US 2024/0307651 A1
(43) Pub. Date: Sep. 19, 2024

(54) Devices, Systems, and Methods for Monitoring and Managing Resilience

(71) Applicant: Innsightful, Inc., Ann Arbor, MI (US)

(72) Inventor: Farzad Ehsani, Sunnyvale, CA (US)

(21) Appl. No.: 18/573,098

(22) PCT Filed: Oct. 21, 2022
(86) PCT No.: PCT/US2022/047348

§ 371 Date: Dec. 21, 2023

Related U.S. Application Data
(60) Provisional application No. 63/270,733, filed on Oct. 22, 2021.

(51) Int. Cl. A61M 21/00; A61B 5/0531; A61B 5/16

(52) U.S. Cl. CPC A61M 21/00; A61B 5/0531; A61B 5/165; A61M 2021/0027; A61M 2021/005

(57) ABSTRACT

A device, system, and method for assessing and managing resilience of an individual, particularly in regard to levels of stress, anxiety, and hardship the individual is capable of handling. A system is configured to evaluate physiological data of the individual and to estimate a resilience of the individual based on the evaluation. The resilience may relate to an ability of the individual to manage, or not manage, a stressor based on one or more resilience parameters. The system may intervene to help the individual improve their resilience, for example, if the individual has not coped with and/or recovered from a past stressor, is not coping with and/or recovering from a current stressor, and/or is not prepared to cope with and/or recover from a future stressor.

### CROSS-REFERENCE TO RELATED APPLICATIONS

This application claims priority and benefit to Provisional Patent Application Ser. No. 63/270,733 filed on Oct. 22, 2021.

### TECHNICAL FIELD

The disclosure relates to computer program products, devices, systems, and methods for assessing and managing resilience of an individual. A system is configured to evaluate physiological data of the individual and to estimate a resilience of the individual based on the evaluation. The resilience may relate to an ability of the individual to manage, or not manage, a stressor based on one or more resilience parameters such as a resilience level, a resilience capacity, a resilience depletion rate, and/or a resilience restoration rate. One or more resilience parameters may be particular to the individual, a group of individuals, the stressor, and/or a group of stressors. The disclosure provides improved approaches for monitoring and improving resilience to help individuals cope with and recover from past, present, and future stressors.

### BACKGROUND

Many individuals, both in the U.S. and throughout the world, are unable to effectively manage stressors in their life, which can lead to negative consequences for the individual and the people around them. For example, an individual's inability to cope with and recover from stressors in their personal life can bleed into the individual's professional life and negatively impact their professional performance, and vice versa. If the individual continues to mismanage their stressors, the individual and the people around them, such as family members and coworkers, may be at greater risk of developing health and behavioral problems such as substance abuse, sleep disorders, post-traumatic stress disorder (PTSD), anxiety disorders, aggression, attention deficits, acute stress reaction, adjustment disorders, neglect, maltreatment, and depression. Complications resulting from these and other issues can increase risk for additional problems such as social withdrawal, lower mental and physical well-being, self-harm, and suicide attempt.

Resilience is the ability to cope with and recover from adverse stressful experiences that can lead to behavioral health issues and chronic disease. Among factors that may affect the ability of the individual to manage stressors, resilience is key. Resilience may be considered as the positive outcome of stressful conditions, which contributes to an individual's readiness for future stressors. Resilience can be learned and improved through the acquisition of coping skills prior to exposure to adverse stress, and as part of the recovery process after experiencing harmful stress. Effective resilience in the face of adversity is dependent on individual factors (e.g., positive coping, positive affect, positive thinking, realism, behavioral control, physical fitness, and altruism), family factors (e.g., emotional ties, communication, support, closeness, nurturing, and adaptability), professional environment factors (e.g., positive climate, teamwork, and cohesion), and community factors (e.g., belongingness, cohesion, connectedness, and collective efficacy).

Certain individuals and professionals, such as armed forces service members, athletes, and others, may learn to manage and increase their resilience through training, self-awareness, and developing an understanding of their own cognition. These and other individuals mentally and physically prepare themselves for stressful periods that require enormous amounts of energy expenditure. However, despite the known benefits of having a higher resilience, many individuals are ill-equipped to monitor and improve their resilience for management of their stressors.

The concept of a mobile phone battery is a useful analogy to explain resilience. Some activities (e.g., presentations, meeting with difficult people, math class) are more stressful and drain a person's battery fast, while other activities (exercise, mindfulness, spending time with friends or family) recharges their resilience “battery.” By measuring existing resilience battery level and stress expenditure, we can provide individuals with a clear picture of when they will run out of battery and when they should recharge.

A number of software-based approaches exist for developing mindfulness for reducing chronic stress and anxiety, however, these existing approaches do not provide any psychotherapeutic features, or have had only one principle of therapy, and many did not integrate clinical experts. While some mindfulness-based interventions can potentially reduce symptoms in subclinical anxiety, generalized anxiety disorder (GAD), and social anxiety disorder, user compliance with these interventions is often low, and most software applications are unable to provide targeted intervention. For many mobile health software applications, psychotherapeutic features are limited and focus on primitive mood tracking and recording thoughts or emotions without providing the user any guidance to address issues. While some approaches may measure symptoms and provide access to therapists, the cost of access is often too high and the quality of services is variable or unreliable. Additionally, fully automated software solutions tend to lack personalization and individualized content, leaving the user without any tools to assist with their respective issues. Furthermore, none of these solutions can provide actionable intelligence on resilience on a timely basis.

Existing solutions for managing stress, as well as mental health and behavioral health, are not adequately meeting the growing demand for these services. Limited availability of many therapists and psychiatrists leads many patients to forego treatment altogether, which can result in a patient experiencing significant mental health crises while waiting to see a health professional. Additional factors, such as transportation cost or availability, and lack of acceptable health insurance, further complicate traditional treatment approaches. The effect is a high healthcare and economic burden on both caretakers and patients. As a result, many people facing new stressors are unable to cope and these stressors may turn into chronic mental or behavioral health issues. Developing resilience—a coping mechanism for stress—can decrease the chances that the person develops chronic stress.

Accordingly, there is a need for intelligent real-time automated monitoring of resilience. This organized monitoring will allow for intervention with individuals for the development and use of improved resilience, which will, in turn, assist with the management of stressors, behavioral health issues, and mental health issues.

The present disclosure addresses this unmet need.

### SUMMARY

The disclosure provides an end-to-end platform to monitor and manage an individual's mental and psychological resilience. A system is configured to analyze data to evaluate the individual's moment-to-moment resilience in response to both positive and negative stressors, as well as provide feedback to the individual to help the individual recognize their reserve of resilience, to ultimately try to improve their usage of resilience to cope with stressors in a healthy and productive manner. The platform can be utilized by healthcare providers for diagnosis, non-contact therapeutics, monitoring, and population management. These approaches within the platform may be adapted according to individuals' needs in order to make better use of available healthcare resources. The platform may also be used to monitor at-risk patients, concerning any health condition, as well as those who have received a diagnosis and require remote or at-home monitoring. In certain instances, the approaches in the platform allow intervention for health care management. The approaches disclosed herein also provide many benefits for people with chronic disease conditions as they reach mental and physical exhaustion more quickly.

The disclosure provides approaches for estimating resilience in an individual, or a group of individuals, based on physiological data (e.g., biosensor data) that corresponds to a physiological state of the individual or the group of individuals. The estimation of resilience may involve one or more resilience parameters, such as a resilience level, a resilience capacity, a resilience depletion rate, and/or a resilience restoration rate-one or more of which may be particular to the individual or group of individuals based on their profession, culture, or other social similarities. Specific approaches may utilize a biosensor device, a biological signal (e.g., “biosignal”) event detection artificial intelligence (AI) engine, an automated conversation agent, real-time connectivity with health care providers, a regulatory-compliant (e.g., HIPAA-compliant) backend, or any combination thereof for real-time resilience monitoring, assessment, and in certain instances, stressor management and intervention.

A system for monitoring an individual, the system including a biosensor device, the biosensor device configured to collect physiological information that corresponds to a physiological state of the individual, a processor, said processor in communication with said biosensor device, the biosensor device configured to transmit said information to said processor, the processor configured to receive information from said biosensor device, said processor configured to: receive physiological data from said biosensor device that corresponds to a physiological state of the individual, compute a resilience parameter based on the received physiological data from said biosensor device, and estimate a resilience of the individual, based on the computed resilience parameter, that relates to an ability of the individual to manage one or more stressors, wherein the resilience parameter is derived from a resilience level of the individual and a resilience capacity of the individual, wherein the resilience corresponds to the ability to cope with and recover from adverse stressful experiences that can lead to behavioral health issues and chronic disease. In some embodiments, resilience parameter derived from the resilience level and comprises a resilience depletion rate of the individual, or a resilience restoration rate of the individual, or both the resilience depletion rate and the resilience restoration rate. The processor may be further configured to compute a perceived resilience parameter based on the received data and further configure the processor to compute the resilience parameter and/or estimate the resilience based on the perceived resilience parameter.

The processor may utilize a deep learning model to compute the resilience parameter and/or estimate the resilience. The processor may also take the steps of receive data about a state of the individual and/or a perceived ability of the individual to manage the stressor, compute a perceived resilience parameter based on the received data, and update the deep learning model to compute the resilience parameter and/or estimate the resilience based on the perceived resilience parameter. They processor may further be configured to intervene with the individual to improve the resilience if the individual is unable to manage the stressor.

A device for estimating a resilience of an individual, the device including a processor, a biosensor device, said biosensor device configured to detect physiological data, said biosensor device in communication with said processor, said processor configured to receive physiological data, said biosensor device configured to transmit biosensor data, and a non-transitory machine-readable medium comprising instructions that, when executed by the processor, the processor taking a series of steps including: receive physiological data that corresponds to a physiological state of the individual, compute a resilience parameter based on the received physiological data, and estimate the resilience of the individual, based on the computed resilience parameter, that relates to an ability of the individual to manage one or more stressors, and wherein the resilience parameter is derived from a resilience level of the individual and a resilience capacity of the individual. In some embodiments, the resilience parameter derived by resilience level and capacity comprises a resilience depletion rate of the individual, or a resilience restoration rate of the individual, or both the resilience depletion rate and the resilience restoration rate. In some embodiments, the non-transitory machine-readable medium further comprises instructions that, when executed by the processor, cause the processor to: receive data about a state of the individual and/or a perceived ability of the individual to manage the stressor, compute a perceived resilience parameter based on the received data, and further configure the processor to compute the resilience parameter and/or estimate the resilience based on the perceived resilience parameter. In some embodiments, the non-transitory machine-readable medium further comprises instructions that, when executed by the processor, cause the processor to utilize a deep learning model to compute the resilience parameter and/or estimate the resilience. In some embodiments, the non-transitory machine-readable medium further comprises instructions that, when executed by the processor, cause the processor to: receive data about a state of the individual and/or a perceived ability of the individual to manage the stressor, compute a perceived resilience parameter based on the received data, and update the deep learning model to compute the resilience parameter and/or estimate the resilience based on the perceived resilience parameter. In some embodiments, the non-transitory machine-readable medium further comprises instructions that, when executed by the processor, cause the processor to: intervene with the individual to improve the resilience if the individual is unable to manage the stressor.

A method for managing one or more stressors, the method may include the steps of receiving physiological information that corresponds to a physiological state of an individual; computing a resilience parameter based on the received physiological information, and estimating a resilience of the individual, based on the computed resilience parameter, that relates to an ability of the individual to manage the one or more stressors, wherein the resilience parameter is derived from a resilience level of the individual and a resilience capacity of the individual. In some embodiments, the resilience parameter derived by resilience level and capacity comprises a resilience depletion rate of the individual, or a resilience restoration rate of the individual, or both the resilience depletion rate and the resilience restoration rate. In some embodiments, the method may also include the steps of receiving information about a state of the individual and/or a perceived ability of the individual to manage the one or more stressors, computing a perceived resilience parameter based on the received information, further computing the resilience parameter and/or estimating the resilience based on the perceived resilience parameter, and intervening with the individual to improve the resilience if the individual is unable to manage the one or more stressors.

In one aspect, the system utilizes a processor configured to receive physiological data that corresponds to a physiological state of the individual, and compute a resilience parameter based on the received physiological data. The processor then, based on the computed resilience parameter, provides an estimate for the resilience of the individual and how that may relate to an ability of the individual to manage a stressor. The processor may be configured by an execution of instructions, for example instructions encoded on a non-transitory machine-readable medium or another memory. However, it is contemplated that hardware circuitry may be configured to perform the method steps, and/or computer operations, and may be used instead of or in addition to a software-based approach for carrying out method steps and/or computer operations of the disclosure.

In certain implementations, a processor may be configured to perform any or all of the method steps and/or computer operations of the disclosure using a deep learning model which may be based on any suitable algorithm or approach. Exemplary non-limiting approaches include, but are not limited to, an artificial neural net (ANN) having one or multiple layers, a convolutional neural net (CNN), a continuous regression model such as linear or polynomial regression, a support vector machine (SVM), a random forest, and the like.

In another aspect, the system provides methods and operations for assisting an individual with managing a stressor, involving the steps of receiving physiological information or data that corresponds to a physiological state of an individual, computing a resilience parameter based on the received physiological information or data, and estimating a resilience of the individual, based on the computed resilience parameter, that relates to an ability of the individual to manage the stressor. The methods and operations may be performed, in whole or in part, by the individual, a care provider such as a healthcare worker, a psychiatrist, a therapist, and the like. One or more steps of the methods and/or operations may be able to be performed by a system and/or a device of the disclosure. This system and/or device of the disclosure may work in addition to or instead of being able to be performed by one or more persons. In this manner, hybrid person-computer configurations may be used such that certain steps and/or operations may be automated and other steps and/or operations may be performed manually.

The system may implement various functionalities related to monitoring the individual's physiology in order to estimate resilience and report resilience and/or a resilience parameter to the individual or another person or entity, and the like. In certain implementations, the system queries the individual with a conversational agent and/or a user interface about a mental state of the individual and/or a perceived ability of the individual to manage the stressor. The system may receive information or data from the query, compute a perceived resilience parameter based on the received data, and further configure the processor to compute the various resilience parameters and/or estimate the resilience based on the perceived resilience parameter(s). The system may be configured to query the individual to request information about a biosignal event and/or request information about a physical, emotional, and/or cognitive state of the individual. Such queries may use an established scale, such as the Connor-Davidson Resilience Scale (CD-RISC), and/or may use a new or different scale or metric. These approaches allow the individual to self-report a perceived resilience and/or a perceived resilience parameter(s) to enable the system to configure and/or reconfigure the processor and/or a deep learning model of the system for improved resilience estimation.

In certain implementations, the system may intervene with the individual to improve their resilience. For example, the system may provide therapeutic exercises and/or activities to help the individual cope with and/or recover from a past, present, and/or future stressor. Such intervention may occur if it is determined, for example from the physiological data and/or data or information received from a query, that the individual may have a lower resilience than needed for effectively managing the stressor.

In another aspect, the system leverages artificial intelligence (AI) engines to solve the growing market need for continuous, real-time, and remote mental health and resilience monitoring and management. Implementations of the system may utilize multimodal objective biometrics from wearable technology, AI for detection and classification of events, and in at least some instances, the seamless integration of AI workflows with human clinician experts.

In another aspect, the system provides novel biometric-monitoring technologies which provide an effective solution for early detection of high stress situations, that may cause heavy mismanagement in individuals, for the prevention of mental illness, behavioral problems, etc. These technologies allow real-time analysis of biometric and other types of markers (e.g., verbal, behavioral, etc.) tied to stress imbalance, (e.g., an imbalance between eustress, or positive stress, and distress, or negative stress), as well as real-time analysis of biometric and other types of markers tied to stressor mismanagement. Paired with software applications such as mobile applications, these approaches provide solutions for not only detecting distress and rising stress loads (e.g., with the battery as analogy, a faster energy discharge of the battery), but also generating actionable information regarding an individual's behavioral health and capacity for coping with and recovering from stressors (e.g., with the battery as analogy, better battery energy expenditure management and recharge capabilities). As such, implementations of the system may enable just-in-time (JIT) therapeutics and may provide comprehensive approaches for monitoring and encouraging positive or balanced behavioral health, ultimately improving the health and resilience of patients and individuals.

Another object of the system is to provide computer program products, devices, systems, and methods that may be readily employed to benefit at-risk populations and scale a strained healthcare system to benefit those most in need.

Other objects, features and advantages of the system, will become apparent from the following detailed description taken in conjunction with the accompanying drawings.

### BRIEF DESCRIPTION OF THE DRAWINGS

Although the characteristic features of the system will be particularly pointed out in the claims, exemplary implementations of the system and manners in which they may be made and used may be better understood after a review of the following description, taken in connection with the accompanying drawings, wherein like numeral annotations are provided throughout.

FIG. 1A depicts an exemplary biosensor device next to an exemplary heart rate variability (HRV) graph.

FIG. 1B depicts one or more exemplary biosensor devices, next to an exemplary skin conductance response (SCR) graph, showing electrodermal activity (EDA) as a function of time.

FIG. 2A depicts a table of several exemplary tasks that may be performed as part of data collection in a controlled (e.g., laboratory) and/or uncontrolled (e.g., field) setting.

FIG. 2B depicts a table of several exemplary psychological questionnaires that may be used to query individuals as part of data collection in a controlled (e.g., laboratory) and/or uncontrolled (e.g., field) setting.

FIG. 3 depicts a table of several exemplary sources of physiological data that may be used to compute resilience parameters and/or resilience.

FIG. 4 depicts a diagram of an exemplary end-to-end system according to the system.

FIG. 5 depicts a block diagram of a machine in the example form of a computer device or system, with which instructions may be executed to cause the machine to perform any one or more of the operations and/or methodologies of the system, in whole or in part, individually or in combination, in any order of events or steps.

FIG. 6 depicts a diagram of an exemplary embodiment of the system that utilizes physiological data (e.g., ECG, EDA, respiration, accelerometer, temperature, or any combination thereof) and a convolutional neural net (CNN) for characterizing negative affect (NA) stressors.

FIG. 7A depicts a prophetic bar graph of a baseline resilience depletion in response to a first plurality of stressors.

FIG. 7B depicts a prophetic bar graph of an improved resilience depletion in response to a second plurality of stressors.

FIG. 8A depicts a prophetic bar graph of a baseline resilience restoration as a result of a first plurality of activities.

FIG. 8B depicts a prophetic bar graph of an improved resilience restoration as a result of a second plurality of activities.

### DETAILED DESCRIPTION

Reference is made herein to the attached drawings. Like reference numerals may be used in the drawings to indicate like or similar elements of the description. The figures are intended for representative purposes, are not drawn to scale, and should not be considered limiting.

Unless otherwise defined herein, terms and phrases used in connection with the present disclosure shall have the meanings that are commonly understood by those of ordinary skill in the art.

As used in the description and in the claims, the terms “involving” and “involves” do not exclude other elements or steps. Where an indefinite or definite article is used when referring to a singular noun, e.g., “a,” “an,” or “the,” this includes a plural of that noun unless something else is specifically stated. Furthermore, the terms first, second, third, and the like in the description and in the claims, are used for distinguishing between elements and not necessarily for describing a sequential or chronological order. It is to be understood that the terms so used are interchangeable under appropriate circumstances and that the implementations of the disclosure described herein are capable of operation in other sequences than described or illustrated herein.

As used herein, the term “about” refers to the usual error range for the respective value readily known to the skilled person in this technical field. Reference to “about” a value or parameter herein includes and describes implementations that are directed to that value or parameter per se.

As used herein, the term “resilience level” refers to an amount of resilience that is less than or equal to a maximum amount of resilience attainable by an individual or a plurality of individuals and may be considered as analogous to a charge level of a battery.

As used herein, the term “resilience capacity” refers to a maximum amount of resilience attainable by an individual or a plurality of individuals and may be considered as analogous to the full charge capacity of a battery.

The disclosure provides a digital apparatus for measuring moment-to-moment stress and variations in resilience, as well as for providing individualized interventions to cope with stress and increase resilience. The system helps individuals to understand and increase their resilience and provides individualized just-in-time (JIT) techniques to manage stress and increase resilience, especially in preparation for a task that requires high expenditure of physical, mental, and emotional resources. Some activities are more stressful and drain an individual's resilience faster, while other activities are less stressful and/or are regenerative and recharge the individual's resilience for later availability. By measuring the individual's resilience level (e.g., current resilience level, typical resilience capacity, etc.) and stress expenditure and/or exposure, the system provides the individual with an understanding of their available resilience resources which may be used for planning purposes, such as whether, for a given resilience level in view of a given stress load, the individual should rest or begin another stressful activity.) The system helps the individual discern which stressful tasks deplete resilience level by causing adverse stress, whether the individual is ready for a new task, and/or whether the individual needs intervention to manage stress and replenish resilience.

Any of a variety of interventions may be used to manage stress and/or replenish resilience. Acceptance and Commitment Therapy (ACT) has emerged as one of several promising methods to prevent and treat the deleterious effects of negative stress. Others include Cognitive Behavioral Therapy and its variations, Positive Psychology, Human Givens, etc. ACT promotes psychological flexibility through acceptance rather than avoidance of negative emotions, cognitive diffusion (e.g., recognition that a thought is just a thought), present moment awareness (e.g., mindfulness of current experiences), values (e.g., personal, meaningful), and as a guide to action. In implementations, the system may provide JIT, tailored ACT therapy to users as needed.

The disclosure provides a solution for detecting distress and rising stress loads, providing actionable information regarding affective health, and facilitating JIT ACT. The disclosure provides a comprehensive means of monitoring and encouraging positive affective health among individuals and their families, ultimately increasing resilience and readiness.

The disclosure also provides approaches for measuring and evaluating a person's moment-to-moment stress, anxiety, and reservoir of resilience while providing highly personalized suggestions throughout the day to help the user improve their personal and professional performance. Physiological stress is a condition that challenges the homeostasis of an organism. Psychological stress (e.g., affect) is the result of an individual's perception of sufficient resources, both internal (e.g., psychological, cognitive) and external (e.g., work and family), to meet challenges. If an individual feels that they have sufficient resources, then an event is perceived positively (e.g., a champion diver before a dive), but if an individual feels that they lack sufficient resources (e.g., a novice forced to do the same dive), then the event is perceived negatively. An individual's resilience reservoir is depleted faster during negative affect (NA). Individuals with low resilience are more likely to feel more negative and less positive emotions, and they are less able to disengage from emotional situations. Individuals with high resilience can “bounce back” to equilibrium after both positive and negative emotional situations much more quickly than individuals with low resilience.

Development of implementations of the system may involve three steps: data collection, model building, and optimizing intervention for improving overall resilience. For data collection, lab and field data may be collected from individuals and their families (e.g., spouses and adult children) while participants perform daily routine tasks and exercises and then report on their emotions, wellbeing, sleep patterns, and other cognitive and psychological measures. For model building, AI models may be built, based on collected data, to achieve a desired level of accuracy, for example, a mean absolute percentage error of 8% for participant resilience; the data may also be used to model NA. Intervention optimization may include providing feedback, guidance, and JIT activities to users that increase the user's average resilience. Selection of particular activities may be driven by software modules designed to reduce negative stress and increase psychological flexibility and resilience in the user.

An individual's physical, cognitive, and emotional wellbeing may change on an hourly basis, based on somatic factors (e.g., sleep, nutrition, exercise), emotional state (e.g., positive, or negative emotions, impulse control, emotional regulation, sense of purpose, general life satisfaction), and cognition (e.g., attention, executive function). Perceptions of wellbeing and adequate resources may contribute to resilience, which may vary based on the ability of the individual to manage stress and trauma. Performance on tasks requiring focus and attention, when the individual is sleep-deprived with accompanying negative rumination, will be worse compared to an individual who might be less able to complete the tasks but who has good physical health and a positive outlook. Furthermore, there may be a difference between a person's overall resilience and wellbeing and their hour-to-hour resilience. Overall resilience may be a baseline capacity and hourly variations may be based on physical, emotional, and cognitive wellbeing. A scale such as CD-RISC may be used to capture overall resilience score.

In addition to determining resilience score, several proxies will be used to obtain the individual's overall state of physical, emotional, and cognitive wellness. Accelerometry (e.g., duration and intensity of exercise), heart rate variability (HRV), electrodermal activity (EDA), and sleep quality and duration may contribute to modeling physical wellbeing. HRV and EDA correlate with emotional regulation, impulse control, and positive versus negative feelings. Additionally, HRV and EDA have a high correlation with neuroticism, which has been associated with lower resilience. Cognitive skills may be captured primarily via the use of the Stroop Color and Word Test (SCWT). Separate models may be built to determine physical, emotional, and cognitive skillsets. These models may then be combined into a single model to measure resilience, and CD-RISC may be used to obtain the overall resilience level for each participant and to model a convolutional neural network for accuracy.

In certain implementations, the system may utilize existing wearable biosensor technology (e.g., Corsano Health Bracelet). This may facilitate collection of certain biosignals through wireless data collection of EDA, HR, HRV, temperature, and motion data. User queries by the system may be designed to pinpoint root causes of stress events and extract context to support stress detection and mitigation. For example, “It looks like you experienced stress at 3:30 PM today—What happened?” Follow-up questions may be triggered based on the user response. A natural language understanding system may be developed that, based on real-time trigger words, accurately handles many common scenarios.

To validate the system for emotional health, a dataset (e.g., Wearable Stress and Affect Detection (WESAD)) may be used to demonstrate that the algorithms detect, quantify, and distinguish physical and emotional stress. WESAD includes multi-modal data recorded during different affective states, especially during stress. It also provides PA and NA scores using the Positive and Negative Affect Schedule (PANAS) questionnaire. The PA scale measures levels of being active, interested, inspired, strong, excited, proud, enthusiastic, alert, determined, and attentive. NA items measured are distressed, annoyed, guilty, scared, hostile, irritable, ashamed, nervous, jittery, and afraid. For cognitive stress, the SCWT may be used as this measure has been proven to correlate with cognitive control, executive dysfunction, and mood. Therapeutic intervention, such as ACT intervention, may be used to manage mental health and/or behavioral health conditions, including but not limited to generalized anxiety disorder (GAD).

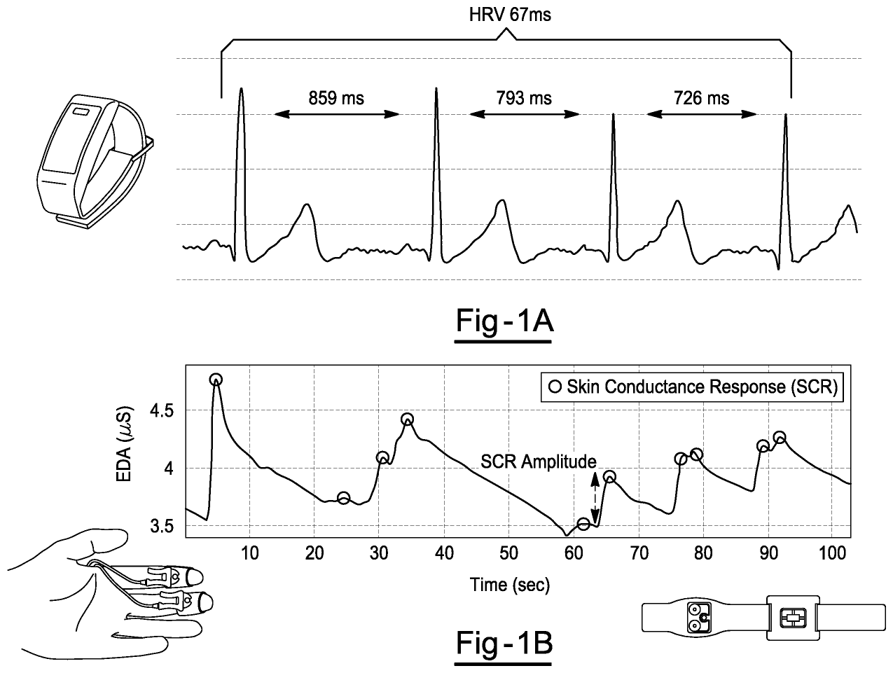

Referring now to FIG. 1A and FIG. 1B, there are depicted an exemplary biosensor device next to an exemplary heart rate variability (HRV) graph (FIG. 1A) and one or more exemplary biosensor devices, next to an exemplary skin conductance response (SCR) graph, showing electrodermal activity (EDA) as a function of time (FIG. 1B). In certain implementations, the system provides a biosensor device (e.g., a wrist worn wearable device, a finger worn wearable device, etc.) and a software application which may be deployed on an individual's device, such as a mobile device, on the biosensor device, and/or on another device such as a networked server. The system provides an end-to-end system to compute resilience parameter(s) and/or estimate resilience, and to detect stress (e.g., distress), stressors (e.g., stressful experiences or events), and anxiety, at the time of, or shortly after, occurrence. The biosensor device may capture heart rate variability (HRV) and/or electrodermal activity (EDA), optionally in combination with other passively collected data sources, as biometric inputs to detect stress and/or compute resilience parameter(s) and/or estimate resilience.

EDA measures skin conductance. Sweat gland activity influences skin conductance and thermoregulation. Sweat glands are regulated by the ANS as well and are modulated during the fight-or-flight response. In particular, the sympathetic branch stimulates sweat glands and elevates sweating. Since the number of active sweat glands increases with sympathetic activation, skin conductivity is proportional to sweat secretion, and a change in skin conductance at the surface reflects the sympathetic activity and provides a non-invasive and sensitive measure of sympathetic activity. EDA, as part of a technology that automatically recognizes psychological stress, anxiety, and depression, is suggested to be a powerful tool both in clinical settings and in daily life.

EDA was traditionally measured by sensors placed on high-density sweat gland areas (e.g., fingers, palms, etc.). The system of the system allows EDA signal collection outside of the laboratory setting with a biosensor device that can continuously measure EDA with designs that integrate sensor contacts. EDA can be assessed by measuring the electrical conductance, resistance, impedance, or admittance of the skin via endosomatic and exosomatic methods to distinguish the tonic and phasic components of the electrical signal. EDA's tonic component, skin conductance level (SCL), is related to the slowly varying skin conductance level, and corresponds to baseline level of skin conductance. SCL may be computed as a mean of several measurements taken during a specific non-stimulation rest period. Thus, SCL is slowly changing and measures general psychophysiological activation, which can vary substantially among individuals. The phasic component is the rapidly fluctuating part of EDA that corresponds to the response to a specific and discrete stimulus, such as when the sudomotor nerve is activated. Generally, the increase in skin conductance starts 1 to 4 seconds after stimulus exposure, and persists for 1 to 3 seconds, allowing different amplitudes to be easily measured.

Difficulties arise when responses occur continually and close together in time, making it difficult to distinguish between individual peak events. Accordingly, the present disclosure additionally relates to novel approaches for extracting overlapping peaks and filtering noise artifacts. In embodiments, these approaches measure raw conductance and apply an algorithm to extract the tonic and phasic components into separate features, for example, Phasic Peaks, Max and Variance, Tonic Peaks, Mean and Variance, and the like. In this manner, the peak events can be characterized, and nervous system activity monitored and quantified.

HRV may be considered an indicator of better general health status, self-regulatory capacity, adaptability, and emotional and stress resilience. Low HRV may be an indicator of abnormal and insufficient adaptation of the autonomic nervous system (ANS) and may be associated with harmful health events, especially when sustained for a prolonged period. A wearable biosensor device may measure HRV on the wrist through photoplethysmography (PPG) input to software to calculate several temporal and spectral parameters. HRV can be measured over different time periods, from less than 5 minutes to 24-hours. Respiration can be estimated from HRV with high accuracy and may further improve system accuracy.

Raw signal EDA measures autonomic changes in skin electrical properties, typically reported in micro siemens. A change in raw value may indicate emotional or cognitive activity. EDA can be split into 2 components: phasic (rapid response) and tonic (slow response). Derived features extracted from each component include minimum, maximum, mean, standard deviation, and peak counts. Phasic peak counts are robust indicators of a stress event.

Quality can be improved by combining with other derived EDA values and non-EDA features. In embodiments, the system involves a functionality configured to detect and classify mental health events, including but not necessarily limited to anxiety, distress, and depression, with a high degree of accuracy. The system may utilize a rule-based algorithm for distress detection based on both EDA and HRV using a ML algorithm. In embodiments, a Tree-based Pipeline Optimization Tool (TPOT) may be utilized, which is an automated machine learning tool that optimizes machine learning pipelines using genetic programming, to build the best model. By using this mixed-methods approach, the shortcomings associated with individual methods have been overcome. In embodiments, the system may accurately detect when the individual is in one of several states, including a calming task, a negative stressor task, and a positive stressor task.

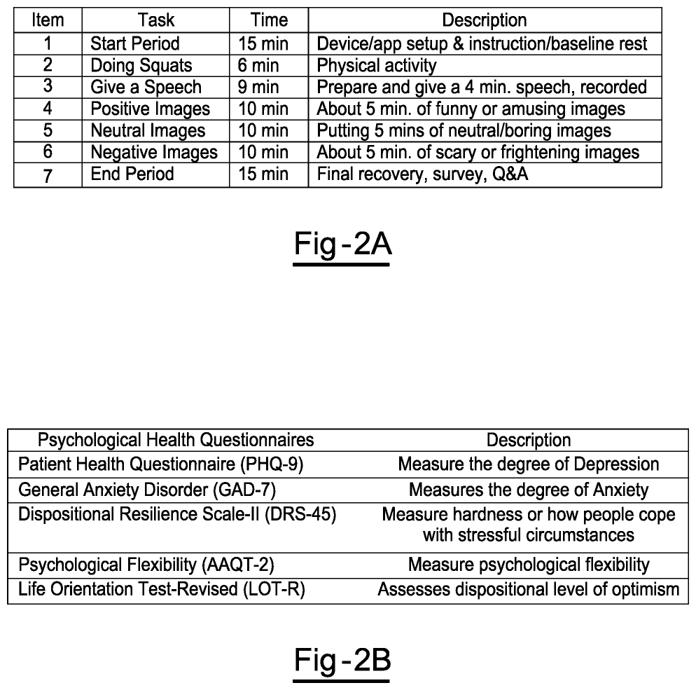

Referring now to FIGS. 2A and 2B, there are depicted a table of several exemplary tasks that may be performed as part of data collection in a controlled (e.g., laboratory) and/or uncontrolled (e.g., field) setting (FIG. 2A) and a table of several exemplary psychological questionnaires that may be used to query individuals as part of data collection in a controlled (e.g., laboratory) and/or uncontrolled (e.g., field) setting (FIG. 2B). In implementations, a data collection tool of the system may present tasks described in FIG. 2A to a user. Lab data may be collected by a research assistant (RA) monitoring data collection and biosignal capture from the participants. At the start of data collection, demographic information (e.g., age, gender, race/ethnicity, etc.), along with medical history including mental health history, current medications, substance use, etc., may be collected. The user may be asked to complete one or more psychological health questionnaires (FIG. 2B) to provide psychological characteristics and to list significant stressors. A sample of blood may be collected to measure peak cortisol level.

Biosignal data collection and analysis may be completed using a biosensor device and a processor configured to receive and analyze physiological data. Data may be collected while individuals complete certain tasks (FIG. 2A) that elicit social, physical, and cognitive stressors. The task order may be randomized to ensure no order dependence. During the 5-minute recovery period at the end of each task, subjects may be given a guided breathing exercise. At the beginning and end of each condition, the individual may complete the PANAS questionnaire. In addition, data collection may continue in the field (e.g., outside the laboratory setting), where the users may wear the device for at least 12 hours per day. Physiological data will be collected, and users may be asked to regularly self-report their stress level and to self-report their stress level whenever the algorithm detects a major stress event. Causes of stress may be queried using a user interface and/or a dialog system (e.g., via text or voice). The queries may include short-form questions. In addition, demographic information, and responses to psychological questionnaires (FIG. 2B), may be used as input features for model building.

Deep learning models, especially convolutional neural networks (CNN), are very effective for a range of applications including emotion classification. CNNs are very useful when applied to unprocessed data and can interpolate the best set of features automatically. This data-driven feature extraction is considered the main advantage of CNNs, especially when using physiological signals where the emotion-related signal features may not be known without prior knowledge. Extracting emotion features from each modality in multi-modal data may require a different type of CNN architecture to capture patterns from these physiological signals and resulting CNN models may be combined at a later stage. In some instances, the physical, emotional, and cognition models may be independent of each other and as a result, three independent models may be built and used to measure the level of physical, emotional, and cognitive wellbeing. These three independent models may be combined into one model for estimating moment-to-moment resilience parameters and/or moment-to-moment resilience. In some instances, the variables may be interdependent, and as a result, a combined model may be built and used, which models resilience directly. In certain implementations, both (independent and interdependent) models may be used to better integrate with an individual's perceived distress and to provide greater resolution to changing anxiety levels. In certain implementations, cognitive, emotional, and physical wellbeing may be modeled separately and a measure (e.g., a mean of these three models) may be used to compute resilience parameters and/or estimate resilience.

In implementations, content may be developed and used that utilize JIT stress reduction techniques such as body scan, mindful seeing and/or listening, self-compassion, diaphragmatic breathing, and/or self-guided imagery, which may reduce negative symptoms of stress. Such approaches may be based on mindfulness and relaxation techniques, cognitive behavioral therapy, and positive psychology. The content may be personalized to each user based on measured (e.g., via physiological data) effectiveness.

In implementations, a baseline perceptual model for resilience NA may be used; for instance, when an individual's perceptual biosignals indicate potential stress (e.g., lowered resilience and heightened NA), the individual may receive a prompt for stress self-reporting similar to the lab test and incident detail (e.g., may be used for providing personalized therapeutics). The individual may respond to the prompt later if occupied, and/or the individual may provide a daily summary of stressors.

In implementations, a controlled condition used for data collection may include a laboratory setting, a medical setting, or another setting which enables controlled observation of the individual. In implementations, a trained device or system may exhibit improved evaluation of stress and resilience. In addition, or in the alternative, such trained devices or systems may benefit other users as well, for example, by constructing or updating a model for all or a subset of users of the system. The subset of users may include, for example, other users who exhibit similar responses to a set of tasks or stimuli and/or other users who have a similar resilience. The subset of users may include geographic subsets, socioeconomic subsets, or other subsets with suitable characteristics.

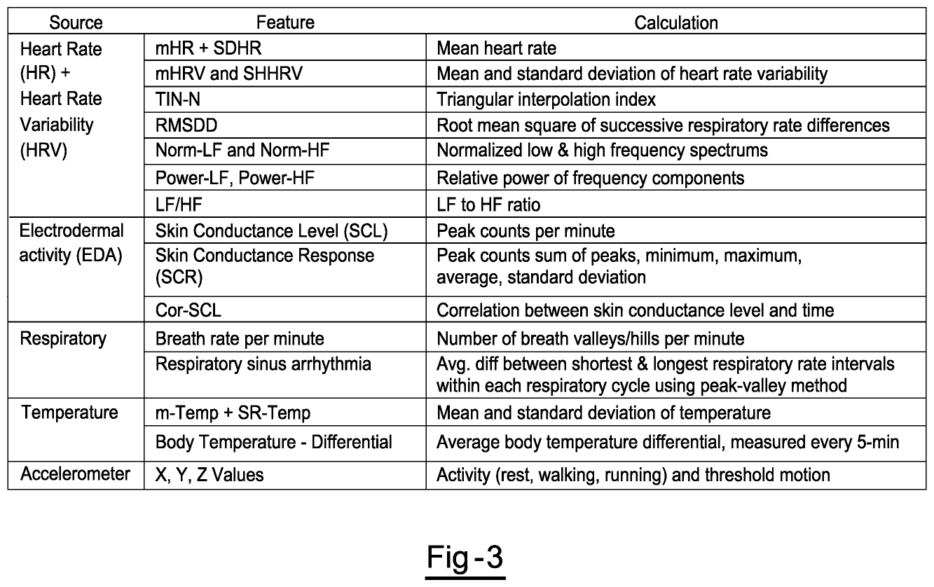

Referring now to FIG. 3, there is depicted a table of several exemplary sources of physiological data that may be used to compute resilience parameters and/or resilience. In implementations, this data may be augmented with psychological health questionnaire (e.g., from FIG. 2B) for better modeling. A plurality of data domains may be used to extract features that may be correlated to physiological stress both in the lab and in the field data collections. Many of these features have been validated in studies including HR/HRV, EDA, respiratory, motion, temperature, and their derivatives. A combination of these features may improve system performance and patient outcome.

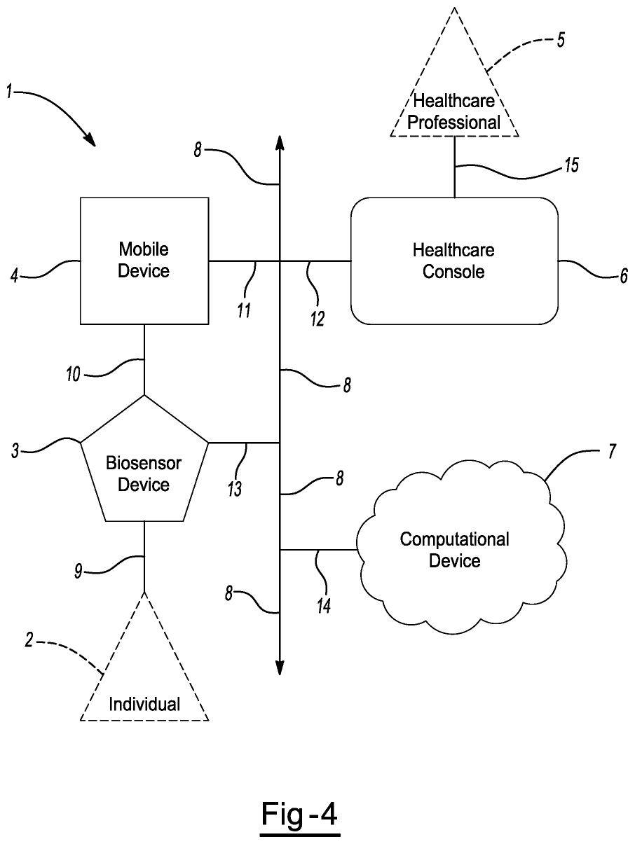

Referring now to FIG. 4, there is depicted a diagram of an exemplary end-to-end system. The embodiment provides a system 1 for stress detection and management, comprising one or more devices (e.g., 3, 4, 6, 7) configured to monitor physiological data of an individual 2 who may be wearing or interacting with a device of the one or more devices. The system 1 may be configured to detect a biosensor event and to characterize the event as correlating with no stress, positive stress, negative stress, or both. In the shown embodiment, the individual 2 may connect with a healthcare professional 5 or another individual capable of aiding the individual 2. In this manner, the individual 2 may communicate with the healthcare professional 5 if needed for delivery of care to the individual 2.

Connections 8, 9, 10, 11, 12, 13, 14, and 15 may include, but are not necessarily limited to, any known wired or wireless connection type suitable for transmission of data, such as digital data. Generally, these connections are necessary to enable components of the system (e.g., a biosensor device 3, a mobile device 4, a healthcare console 6, and a computational device 7) to intercommunicate. Exemplary connection types that may be suitable for one or more of connections 8, 9, 10, 11, 12, 13, 14, and 15 include ethernet or other wired connection type, WiFiR connectivity, Bluetooth® connectivity, or other electromagnetic or radio wave connectivity as needed according to an embodiment.

In the shown embodiment, the individual 2 is wearing a biosensor device 3 and is operably connected thereto by connection 9. Connection 9 may be representative of a physical connection, as may be utilized with attachment of the biosensor device 3 to the wearer 2, but particularly, connection 9 may be representative of an operable connection configured to enable observation of the biological property of the wearer 2 by the biosensor device 3. For example, connection 9 may include, but is not necessarily limited to, a wire connected to a skin conductance sensor for EDA measurement, an optical connection for performing pulse oximetry or heart rate detection for HRV measurement, or a combination thereof. In like manner, and consistent with embodiments of the connection 9, the biosensor device 3 may be configured to read one or more biological properties to obtain physiological data, including but not necessarily limited to a heart rate (HR), a heart rate variability (HRV), an oxygen saturation (SpO2), an electrodermal activity (EDA), a breathing rate (BR), a body temperature (BT), a movement, and any combination thereof. In embodiments, the biosensor device 3 involves an optical data acquisition system that is configured to perform pulse oximetry (for SpO2 determination) and heart rate detection (for HR or HRV determination). In embodiments, the biosensor device 3 may be configured for placement on a wrist, a finger, or an ear of the wearer. In this manner, the blood oxygen level may be optically determined, along with other parameters accessible from these body parts, according to need. Further, it may be anticipated that the addition of new sensors (e.g., sensors coming onto the market, such as sweat sensors configured to analyze the chemical content of sweat, and/or blood sugar sensors as may be present in an existing wearable device such as the Apple Watch series 7 and/or later series) may be used, in addition to these devices or in the alternative, to implement or improve the analyses disclosed herein.

In the shown embodiment, the one or more devices involves a mobile device 4 and the biosensor device 3, wherein the mobile device 4 is operably connected to the biosensor device 3 by operable connection 10. Operable connection 10 may include any connection suitable for electronic data transmission, for example, a wired connection, or one or more wireless data connections. In embodiments, the biosensor device 3 is operably connected to the mobile device 4 by the operable connection 10, wherein the operable connection 10 is involved of a Bluetooth R wireless connection. In this manner, the biosensor device 3 is sufficiently compatible with existing mobile devices.

In the shown embodiment, the mobile device 4 is connected to a network 8, such as the world-wide web or internet, by an operable connection 11. Operable connection 11 may include any connection type suitable for data transmission, including a wired connection or a wireless connection. Operable connection 11 may be direct or indirect, and may proceed through any number of routers, cell towers, satellites, and the like. In this manner, the overall system may be configured for use with any of a variety of different network designs or topologies, according to need and availability of network infrastructure in an embodiment. As ordinarily understood, network 8 may include but is not necessarily limited to a trans-continental network such as the world-wide web or internet. Network 8 may include any number of connection points, as would be understood by a person having ordinary skill in the art.

In the shown embodiment, the biosensor device 3 is operably connected to the network 8 by operable connection 13. In embodiments, the biosensor device 3 may contain one or more networking functionalities, such as one or more wireless transceivers, to enable this functionality. In embodiments, the operable connection 10, the mobile device 4, and the operable connection 11 may not be needed for full functionality of the system 1. For example, in embodiments, the biosensor device may include a non-transitory machine-readable medium containing data storage and machine-executable instructions thereon which may effectively replace the need for the mobile device 4. In this manner, the number of components of the system 1 may be reduced or maintained at a level manageable for a particular need, such as an individual 2 that may not utilize the mobile device 4 to use the system.

In embodiments, the system 1 involves the computational device 7, and the computational device 7 may involve a networked server, operably connected to the mobile device 4 and configured to execute a machine learning (ML) algorithm to evaluate a detected event. The computational device 7 may be operably connected to the mobile device 4 by the network 8, and by at least network connection 14. Generally, network connection 14 may be any wired or wireless connection suitable for data transfer, and in this manner, the computational device 7 may be connected to the network 8 and transmit and receive data as needed.

In embodiments, one or more devices, such as the computational device 7, may include a module for event-driven activities, a module for natural language understanding (NLU), a module for speech to text (ASR), or a combination thereof. These modules may be provided as executable computer algorithms, in the form of machine-readable instructions stored in a non-transitory machine-readable medium of the computational device 7. As the system 1 operates, requests from the mobile device 4 or the biosensor device 3 may be processed and responses produced accordingly. The computational device 7 may include any suitable server or cloud-based networking infrastructure, or equivalent combination of computational hardware, which effectively enables cloud-based computing and communication.

In embodiments, one or more of the one or more devices (3, 4, 6, 7) involves a history of the individual 2, wherein the history involves data that pertains to a previous symptom, a previous intervention, an efficacy of a previous intervention, or a combination thereof. In this manner, the system 1 may learn from previous interactions with the individual 2 to anticipate symptoms and improve interventions. In embodiments, the computational device 7 may include a data structure for storage and retrieval of therapy lessons, and a data structure for storage and retrieval of user data, sensor data, audio files, chat logs, or a combination thereof. In this manner, the history of the individual may be stored on the computational device.

The healthcare console 6 may be involved of any suitable networked computing device, such as a mobile device (i.e., mobile phone, tablet, etc.), or a personal computer or workstation. The healthcare console 6 may be configured to enable the healthcare professional 5, such as a doctor, a psychiatrist, a therapist, and the like, to chat with the individual 2 and provide therapy recommendations based on a current situation in view of the history of the individual 2. The healthcare console 6 may be connected to the network 8 by connection 12, which may be a wired or a wireless data transfer connection.

In embodiments, the AI engine runs on the smartphone and executes time series event detection using a classification engine. The AI engine may classify a biosignal event, if possible, so that predetermined logic can be executed. If there is any doubt of the classification, then the individual may be queried to identify the type(s) of stress correlated with the event. This approach allows the logic to be easily trained on tagged data, and a limited amount of verification may be necessary. In embodiments, the automated conversational agent may provide tagging of events (e.g., from the individual) which can facilitate training of the model(s) of the system and provide customization of the system to the individual or a group of individuals. In this manner, the system may be more effectively trained, and may be smaller, more lightweight, and generally improved compared to data-heavy approaches.

The classification engine may be configured to capture multiple inputs and produce secondary data sets (e.g., combinations) that rapidly increase the complexity of the calculations. The classification engine may be assisted by the automated conversation agent, with input from the individual, to classify the event as needed. In embodiments, the classification engine may include a support vector machine (SVM), which is a non-neural network algorithm for manipulating data by adding dimensional information to classify data. In embodiments, the classification engine may include a random force algorithm. In embodiments, the system may provide a time-series analysis.

In embodiments, the system provides a simple, easy-to-use interface for communicating with a health care provider, with either live chat or with the AI-driven mode. This enables direct communication with parties to request additional information, answer questions, or offer therapeutic suggestions. In embodiments, the secure backend data architecture summarizes user information (plots, charts, event logs) concerning progress and outcomes to the care team. Additionally, it flags potential issues based on the results. It also allows healthcare providers to set reminders for remote users and schedule questionnaires. Access is secure, restricted, and logged. All data and logs are encrypted in flight and on the back-end database. In this manner, security and regulatory compliance are attained.

The system provides a strong foundation that can be easily scaled to address remote healthcare monitoring during a crisis, as well as future needs. To perform monitoring a variety of types and combinations of biological signals may need to be captured. A reference design hardware technology (e.g., as may be provided by Maxim Integrated or Texas Instrument or another provider) can collect a wide array of biological signals including HR, HRV, motion, respiratory rate, temperature, and SpO2. The system also may leverage a secondary reference design (e.g., as may be obtained from Philips Research) that integrates electrodermal activity. Coupled with the biological signal front end hardware, the secure mechanism for cloud data transmission and storage facilitates remote data collection, with algorithms to scale and reduce data living both on a smart phone application, as well as in the cloud.

In embodiments, the combination of one or more biological signals to be evaluated may evolve as data becomes available. This aligns with a major technical innovation of the system, to adaptively change the combinations and types of biological signals that can be recorded. A baseline for these indicators can be established before stress events appear. For example, a good model of a user's body temperature and level of tiredness at various times during the day may be obtained. The system can then verify the authenticity of detected events (e.g., coughing, tiredness), and ask for indicators of other symptoms (like gastrointestinal issues) on a regular basis. If there is any indication of a crisis, the individual can be put in touch with frontline telehealth providers or provided with just-in-time and other digital therapies such as videos, games, or interactive content.

In embodiments, the system involves one or more biosensor devices which generate physiological data by monitoring a biological property of the individual.

Methods and operations of the system may be performed, in whole or in part, by a mobile device operably connected with the one or more biosensor devices. In embodiments, the mobile device may include appropriate hardware necessary or sufficient for performing the method in whole or in part, as would be understood by a person having ordinary skill in the art. Exemplary hardware may include a processor, a non-transitory machine-readable medium, a speaker, a microphone, and a human-readable display. In embodiments, one or more logics may be stored on the non-transitory machine-readable medium of the mobile device and executed by the processer of the mobile device when the method is performed. In embodiments, artificial intelligence (AI) algorithms may be trained and validated to detect negative stress and measure resilience based on biosensor physiological data. The software application may compute resilience parameter(s) and/or estimate resilience and/or provide one or more just-in-time activities, such as positive activities and/or therapeutic interventions, to sustain and/or improve the individual's resilience in real-time. Furthermore, the system provides a speech interface that may utilize AI and natural language processing (NLP) to “interview” or query the individual, collecting subjective data that relates to that individual. These interviews may request information about a state of the individual and/or a perceived ability of the individual to manage the stressor. Results of these AI/NLP highly personalized interviews and biometrics may inform short-term and long-term models and/or processor configurations for computing resilience parameter(s) and/or estimating resilience and/or providing therapeutic interventions to improve resilience and better manage stress and anxiety, as well as other behavioral and/or mental health issues as may occur over time.

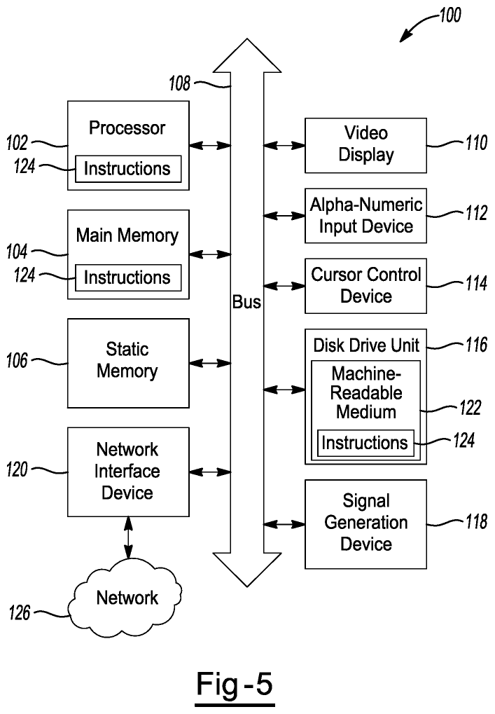

Referring now to FIG. 5, there is depicted a block diagram of a machine in the example form of a computer device or system 100 with which instructions 124 may be executed to cause the machine to perform any one or more of the operations and/or methodologies of the system, in whole or in part, individually or in combination, in any order of events or steps. In embodiments, the machine operates as a standalone device, or can be connected (e.g., networked) to other machines. In a networked deployment, the machine can operate in the capacity of a server or a client machine in server-client network environment, or as a peer machine in a peer-to-peer (or distributed) network environment. The machine can be a personal computer (PC), a tablet PC, a set-top box (STB), a personal digital assistant (PDA), a cellular telephone, a web appliance, a network router, switch, or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein.

The example computer system 100 includes a processor 102 (e.g., a central processing unit (CPU), a graphics processing unit (GPU), or both), a main memory 104, and a static memory 106, which communicate with each other via a bus 108. The computer system 100 can further include a video display 110 (e.g., a liquid crystal display (LCD) or a cathode ray tube (CRT)). The computer system 100 also includes an alpha-numeric input device 112 (e.g., a keyboard or a touch-sensitive display screen), a user interface (UI) navigation (or cursor control) device 114 (e.g., a mouse), a disk drive unit 116, a signal generation device 118 (e.g., a speaker), and a network interface device 120.

The disk drive unit 116 includes a machine-readable medium 122 on which are stored one or more sets of data structures and instructions 124 (e.g., software) embodying or utilized by any one or more of the methodologies or functions described herein. The instructions 124 can also reside, completely or at least partially, within the main memory 104 or within the processor 102, or both, during execution thereof by the computer system 100, with the main memory 104 and the processor 102 also constituting machine-readable media.

While the machine-readable medium 122 is shown in an example embodiment to be a single medium, the term “machine-readable medium” can include a single medium or multiple media (e.g., a centralized or distributed database, and/or associated caches and servers) that store the one or more instructions 124 or data structures. The term “machine-readable medium” shall also be taken to include any tangible medium that is capable of storing, encoding, or carrying instructions 124 for execution by the machine and that cause the machine to perform any one or more of the methodologies of the present disclosure, or that is capable of storing, encoding, or carrying data structures utilized by or associated with such instructions 124. The term “machine-readable medium” shall accordingly be taken to include, but not be limited to, solid-state memories, and optical and magnetic media. Specific examples of machine-readable media 122 include non-volatile memory, including by way of example semiconductor memory devices (e.g., erasable programmable read-only memory (EPROM), electrically erasable programmable read-only memory (EEPROM), and flash memory devices; magnetic disks such as internal hard disks and removable disks; magneto-optical disks; and CD-ROM and DVD-ROM disks).

The instructions 124 can be transmitted or received over a communication network 126 using a transmission medium. The instructions 124 can be transmitted using the network interface device 120 and any one of a number of well-known transfer protocols (e.g., HTTP). Examples of communication networks include a local area network (LAN), a wide area network (WAN), the Internet, mobile telephone networks, plain old telephone (POTS) networks, and wireless data networks (e.g., Wi-Fi and WiMax networks). The term “transmission medium” shall be taken to include any intangible medium that is capable of storing, encoding, or carrying instructions 124 for execution by the machine, and includes digital or analog communications signals or other intangible media to facilitate communication of such software.

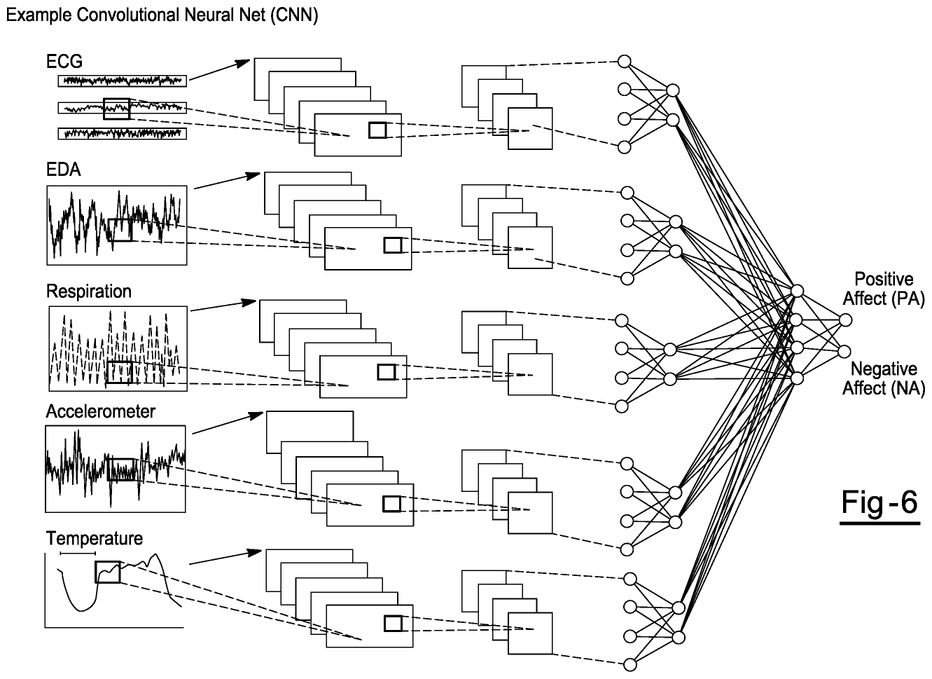

Referring now to FIG. 6, there is depicted a diagram of an exemplary embodiment of the system that utilizes physiological data (e.g., ECG, EDA, respiration, accelerometer, temperature, or any combination thereof) and a CNN for classifying events as being positive affect (PA) or negative affect (NA). In embodiments, the quantitative processed features from the wearable device may be used to build a physiological stress model. In embodiments, simple machine learning algorithmic classifiers, such as support vector machine(s) and/or random forest(s), and more complex models if needed, such as a multi-layer NN network, may be utilized. In embodiments, a self-learning optimizer such as the Tree-Based Pipeline Optimization Tool (TPOT) may be used to apply multiple preprocessing steps and machine learning algorithms.

It may be more effective to integrate with an individual's perceived distress to provide greater resolution to changing anxiety levels. In embodiments, one implementation of the system would be to model the accumulation and decay of stress over time, of either positive stress or negative stress, or both. Another implementation would be to model the stress as two continuous regressions. One regression would be the level of stress (from none to high), and another regression would be the sign or polarity. In embodiments, it is possible to accurately model when the subject is in one of three states from laboratory data (including, for example, a calming task, a distress-causing task, and a eustress-causing task).

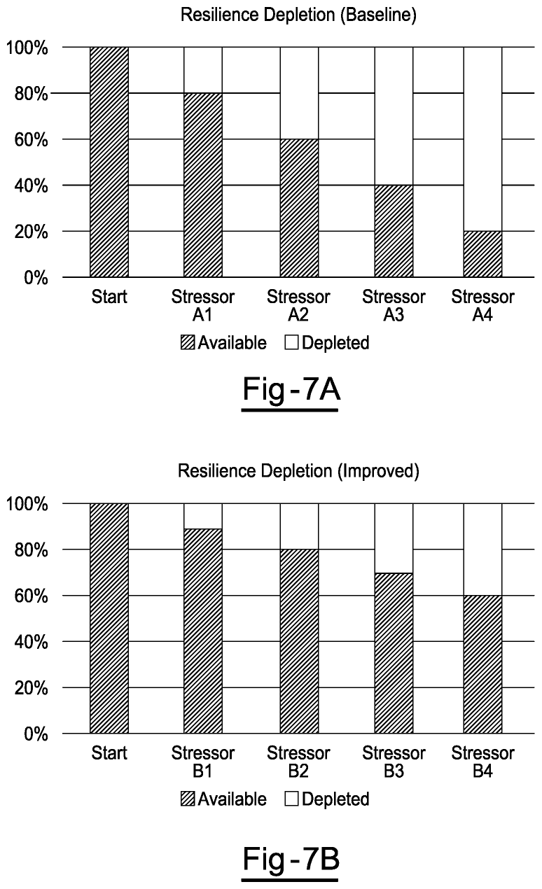

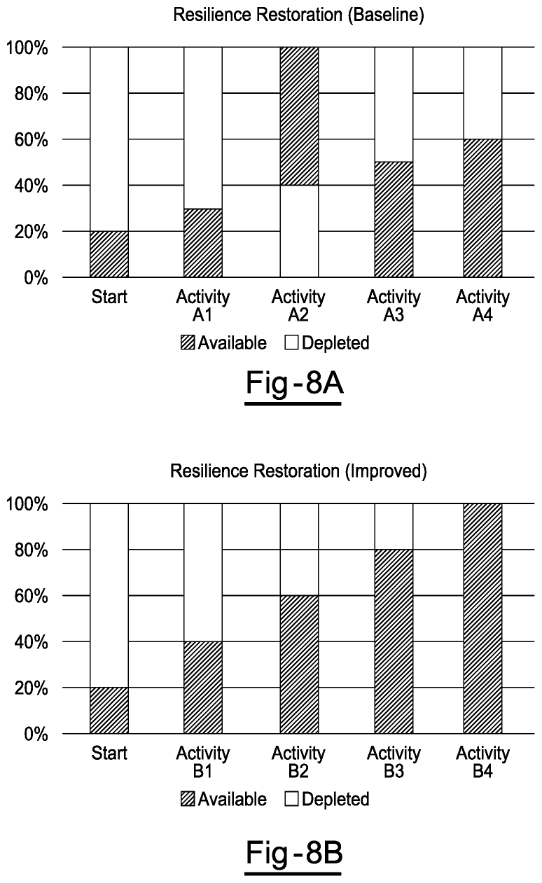

Referring now to FIGS. 7A, 7B, 8A, and 8B, there are depicted prophetic bar graphs of a baseline resilience depletion in response to a first plurality of stressors (FIG. 7A), an improved resilience depletion in response to a second plurality of stressors (FIG. 7B), prophetic bar graphs of a baseline resilience restoration as a result of a first plurality of activities (FIG. 8A) and a baseline resilience restoration as a result of a second plurality of activities (FIG. 8B). Resilience may be considered as a steady state quantity that is capable of decreasing and increasing according to different factors such as stress exposure as well as the quantity and quality of healing activities such as rest and/or therapeutic intervention to regain resilience lost (e.g., temporarily lost) to stressors. Understanding how an individual's resilience changes in response to these and other factors may facilitate an understanding of the individual's strengths and weaknesses and help identify areas that need improvement.

For example, before a use of the system, an individual may have a baseline for resilience depletion in response to a plurality of stressors (FIG. 7A; Stressors A1-A4). Stressors A1, A2, A3, and A4 may each be the same stressor or may be different stressors each with about the same impact on the individual's available resilience. At start, the individual may have about 100% available resilience, then after exposure to Stressor A1, may have about 80% resilience, then after exposure to Stressor A2, may have about 60% resilience, etc., until resilience is mostly or completely depleted.

If the individual is more sensitive to the stressors, then resilience may be depleted more per stressor, and similarly, if the individual is less sensitive to the stressors, then resilience may be depleted less per stressor. As such, a goal of the system may be to lower the sensitivity of the individual to the stressor by training the individual to lower their perceived significance of the stressor and thereby lower the impact the stressor has on the individual's mental and/or physical equilibrium and help preserve their resilience for other stressors.

If the individual improves their resilience by lowering their sensitivity to the stressor, then they may be able to handle more exposures to the stressor without depleting their resilience as rapidly. For example, the individual may have an improved resilience depletion in response to a plurality of stressors (FIG. 7B; Stressors B1-B4). Stressors B1, B2, B3, and B4 may be the same as and/or correspond to Stressors A1, A2, A3, A4, but may be perceived differently by the individual after their improvement. Again, these stressors may each be the same stressor or may be different stressors each with about the same impact on the individual's available resilience. At start, the individual may have about 100% available resilience, then after exposure to Stressor B1, may have about 90% resilience, then after exposure to Stressor B2, may have about 80% resilience, etc., and in this manner, the improved individual's resilience may be more slowly depleted and thereby preserved for managing different or more serious stressors the individual may encounter thereafter.

In another example, before a use of the system, an individual may have a baseline for resilience restoration in response to a plurality of activities (FIG. 8A; Activities A1-A4). Activities A1, A2, A3, and A4 may each be the same activity or may be different activities each with about the same impact on restoring the individual's available resilience (e.g., JIT therapy, ACT therapy, etc.). At start, the individual may have about 20% available resilience, then after Activity A1, may have about 30% resilience, then after Activity A2, may have about 40% resilience, etc., until resilience is mostly or completely restored.

If the individual benefits more from the activity, then resilience may be restored more per activity, and similarly, if the individual benefits less from the activity, then resilience may be restored less per activity. As such, a goal of the system may be to increase the benefit to the individual from the activity by training the individual and/or the system to increase the perceived significance of the activity and/or optimize selection of an effective activity to help stabilize, improve the individual's mental and/or physical equilibrium and help restore resilience for other stressors.

If the individual benefits more from the activity, then they may require fewer interventional activities to restore their resilience for other stressors. For example, the individual may have an improved resilience restoration in response to a plurality of activities (FIG. 8B; Activities B1-B4). Activities B1, B2, B3, and B4 may be the same as and/or correspond to Activities A1, A2, A3, A4, but may be perceived differently by the individual after

their improvement, or may be different from Activities A1, A2, A3, A4 and may be selected or optimized for improved effectiveness. The activities may provide about the same benefit to the individual's available resilience. At start, the individual may have about 20% available resilience, then after exposure to Activity B1, may have about 40% resilience, then after exposure to Activity B2, may have about 60% resilience, etc., and in this manner, the individual's resilience may be more quickly restored and thereby preserved for managing different or more serious stressors the individual may encounter thereafter.

The foregoing descriptions of specific implementations have been presented for purposes of illustration and description. They are not intended to be exhaustive or to limit the system to the precise forms disclosed, and modifications and variations are possible in view of the above teaching. The exemplary implementations were chosen and described to best explain the principles of the system and its practical application, to thereby enable others skilled in the art to best utilize the system and its implementations with modifications as suited to the use contemplated.

With respect to the description provided herein, it is submitted that the optimal features of the system include variations in size, materials, shape, form, function, manner of operation, assembly, and use. All structures, functions, and relationships equivalent or essentially equivalent to those disclosed are intended to be encompassed by the system. It is noted that the terms “substantially” and “about” may be utilized herein to represent the inherent degree of uncertainty that may be attributed to any quantitative comparison, value, measurement, or other representation. Any values that may be modified by such terminology are also part of the teachings herein. For example, if a teaching recited “about 10,” the skilled person should recognize that the value of 10 is also contemplated.

These terms are also utilized herein to represent the degree by which a quantitative representation may vary from a stated reference without resulting in a change in the basic function of the subject matter at issue.

As used herein, unless otherwise stated, the teachings envision that any member of a genus (list) may be excluded from the genus; and/or any member of a Markush grouping may be excluded from the grouping.

Unless otherwise stated, any numerical values recited herein include all values from the lower value to the upper value in increments of one unit provided that there is a separation of at least 2 units between any lower value and any higher value. As an example, if it is stated that the amount of a component, a property, or a value of a process variable such as, for example, temperature, pressure, time and the like is, for example, from 1 to 90, preferably from 20 to 80, more preferably from 30 to 70, it is intended that intermediate range values such as (for example, 15 to 85, 22 to 68, 43 to 51, 30 to 32 etc.) are within the teachings of this specification. Likewise, individual intermediate values are also within the present teachings. For values which are less than one, one unit is considered to be 0.0001, 0.001, 0.01 or 0.1 as appropriate. These are only examples of what is specifically intended and all possible combinations of numerical values between the lowest value and the highest value enumerated are to be considered to be expressly stated in this application in a similar manner. As can be seen, the teaching of amounts expressed as “parts by weight” herein also contemplates the same ranges expressed in terms of percent by weight. Thus, an expression in the Detailed Description of the System of a range in terms of at “‘x’ parts by weight of the resulting polymeric blend composition” also contemplates a teaching of ranges of same recited amount of “x” in percent by weight of the resulting polymeric blend composition.”

Unless otherwise stated, all ranges include both endpoints and all numbers between the endpoints. The use of “about” or “approximately” in connection with a range applies to both ends of the range. Thus, “about 20 to 30” is intended to cover “about 20 to about 30”, inclusive of at least the specified endpoints.

The term “consisting essentially of” to describe a combination shall include the elements, ingredients, components or steps identified, and such other elements ingredients, components or steps that do not materially affect the basic and novel characteristics of the combination. The use of the terms “comprising” or “including” to describe combinations of elements, ingredients, components or steps herein also contemplates embodiments that consist essentially of, or even consist of the elements, ingredients, components or steps.

Plural elements, ingredients, components or steps can be provided by a single integrated element, ingredient, component or step. Alternatively, a single integrated element, ingredient, component or step might be divided into separate plural elements, ingredients, components or steps. The disclosure of “a” or “one” to describe an element, ingredient, component or step is not intended to foreclose additional elements, ingredients, components or steps. All references herein to elements or metals belonging to a certain Group refer to the Periodic Table of the Elements published and copyrighted by CRC Press, Inc., 1989. Any reference to the Group or Groups shall be to the Group or Groups as reflected in this Periodic Table of the Elements using the IUPAC system for numbering groups.

While particular embodiments have been illustrated and described herein, it should be understood that various other changes and modifications may be made without departing from the spirit and scope of the claimed subject matter.

Moreover, although various aspects of the claimed subject matter have been described herein, such aspects need not be utilized in combination.

It is therefore intended that the appended claims (and/or any future claims filed in any corresponding application) cover all such changes and modifications that are within the scope of the claimed subject matter.

1) A system for monitoring an individual, comprising:
a biosensor device, the biosensor device configured to collect physiological information that corresponds to a physiological state of the individual:
a processor, said processor in communication with said biosensor device, the biosensor device configured to transmit said information to said processor, the processor configured to receive information from said biosensor device, said processor configured to:
receive physiological data from said biosensor device that corresponds to a physiological state of the individual;
compute a resilience parameter based on the received physiological data from said biosensor device; and
estimate a resilience of the individual, based on the computed resilience parameter, that relates to an ability of the individual to manage one or more stressors;
wherein the resilience parameter is derived from a resilience level of the individual and a resilience capacity of the individual
wherein the resilience corresponds to the ability to cope with and recover from adverse stressful experiences that can lead to behavioral health issues and chronic disease.

2) The system of claim 1, wherein the resilience parameter derived from the resilience level and comprises a resilience depletion rate of the individual, or a resilience restoration rate of the individual, or both the resilience depletion rate and the resilience restoration rate.

3) The system of claim 1, wherein the processor is further configured to:
compute a perceived resilience parameter based on the received data; and
further configure the processor to compute the resilience parameter and/or estimate the resilience based on the perceived resilience parameter.

4) The system of claim 1, wherein the processor is configured to utilize a deep learning model to compute the resilience parameter and/or estimate the resilience.

5) The system of claim 4, wherein the processor is further configured to:
receive data about a state of the individual and/or a perceived ability of the individual to manage the stressor;
compute a perceived resilience parameter based on the received data; and
update the deep learning model to compute the resilience parameter and/or estimate the resilience based on the perceived resilience parameter.

6) The system of claim 1, wherein the processor is further configured to:
intervene with the individual to improve the resilience if the individual is unable to manage the stressor.

7) A device for estimating a resilience of an individual, comprising:
a processor;
a biosensor device, said biosensor device configured to detect physiological data, said biosensor device in communication with said processor, said processor configured to receive physiological data, said biosensor device configured to transmit biosensor data; and
a non-transitory machine-readable medium comprising instructions that, when executed by the processor, the processor taking a series of steps comprising:
receive physiological data that corresponds to a physiological state of the individual;
compute a resilience parameter based on the received physiological data; and
estimate the resilience of the individual, based on the computed resilience parameter, that relates to an ability of the individual to manage one or more stressors;
wherein the resilience parameter is derived from a resilience level of the individual and a resilience capacity of the individual.

8) The device of claim 7, wherein the resilience parameter derived by resilience level and capacity comprises a resilience depletion rate of the individual, or a resilience restoration rate of the individual, or both the resilience depletion rate and the resilience restoration rate.

9) The device of claim 7, wherein the non-transitory machine-readable medium further comprises instructions that, when executed by the processor, cause the processor to:
receive data about a state of the individual and/or a perceived ability of the individual to manage the stressor;
compute a perceived resilience parameter based on the received data; and
further configure the processor to compute the resilience parameter and/or estimate the resilience based on the perceived resilience parameter.

10) The device of claim 7, wherein the non-transitory machine-readable medium further comprises instructions that, when executed by the processor, cause the processor to utilize a deep learning model to compute the resilience parameter and/or estimate the resilience.

11) The device of claim 10, wherein the non-transitory machine-readable medium further comprises instructions that, when executed by the processor, cause the processor to:
receive data about a state of the individual and/or a perceived ability of the individual to manage the stressor;
compute a perceived resilience parameter based on the received data; and
update the deep learning model to compute the resilience parameter and/or estimate the resilience based on the perceived resilience parameter.

12) The device of claim 10, wherein the non-transitory machine-readable medium further comprises instructions that, when executed by the processor, cause the processor to:
intervene with the individual to improve the resilience if the individual is unable to manage the stressor.

13) A method for managing one or more stressors, comprising:
receiving physiological information that corresponds to a physiological state of an individual; computing a resilience parameter based on the received physiological information; and
estimating a resilience of the individual, based on the computed resilience parameter, that relates to an ability of the individual to manage the one or more stressors,
wherein the resilience parameter is derived from a resilience level of the individual and a resilience capacity of the individual.

14) The method of claim 13, wherein the resilience parameter derived by resilience level and capacity comprises a resilience depletion rate of the individual, or a resilience restoration rate of the individual, or both the resilience depletion rate and the resilience restoration rate.

15) The method of claim 13, further comprising:
receiving information about a state of the individual and/or a perceived ability of the individual to manage the one or more stressors;
computing a perceived resilience parameter based on the received information;
further computing the resilience parameter and/or estimating the resilience based on the perceived resilience parameter; and
intervening with the individual to improve the resilience if the individual is unable to manage the one or more stressors.
